(19) United States
(12) Patent Application Publication
(10) Pub. No.: US 2008/0069772 A1
(43) Pub. Date: Mar. 20, 2008

(54) Treatment of transformed or infected biological cells

(75) Inventors: Gernot Stuhler, Tubingen (DE); Helmut Salih, Stuttgart (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Eberhard-Karls-Universitaet Tuebingen Universitaetsklinikum, Tuebingen (DE)

(21) Appl. No.: 11/709,628

(22) Filed: Feb. 22, 2007

Related U.S. Application Data
(63) Continuation-in-part of application No. PCT/EP05/08976, filed on Aug. 19, 2005.
(63) Continuation-in-part of application No. 10/961,320, filed on Oct. 8, 2004.

(30) Foreign Application Priority Data
Aug. 26, 2004 (EP) 04 020 259.0

(51) Int. Cl. A61K 49/00; A61K 31/23; A61K 31/34; A61K 38/02; C12N 15/63; C12Q 1/02; A61P 35/00; A61K 38/16; A61K 39/395

(52) U.S. Cl. 424/9.1; 424/130.1; 424/133.1; 435/29; 435/320.1; 514/12; 514/2; 514/461; 514/552; 514/789; 514/8

(57) ABSTRACT

The present invention relates to a therapeutic or/and diagnostic substance. Furthermore it relates to an expression vector, to a composition comprising the afore-mentioned substance or/and the afore-mentioned expression vector, a method for diagnosing a tumor disease or/and an infectious disease in a living being, as well as to a method for the treatment of a tumor disease or/and of an infection in a living being.

### CROSS-REFERENCES TO RELATED APPLICATIONS

This application is a continuation-in-part of copending international patent application PCT/EP2005/008976 filed on Aug. 19, 2005 and designating the United States, which in turn claims Convention priority from European patent application EP 4 020 259, filed on Aug. 26, 2004, and is also a continuation-in-part of copending U.S. application Ser. No. 10/961,320, filed on Oct. 8, 2004. The respective disclosures of EP 4 020 259 and U.S. Ser. No. 10/961,320 are hereby incorporated herein by reference.

### BACKGROUND OF THE INVENTION

1. Field of the Invention

The present invention relates to a therapeutic or/and diagnostic substance. Furthermore it relates to an expression vector, to a composition comprising the afore-mentioned substance or/and the afore-mentioned expression vector, a method for diagnosing a tumor disease or/and an infectious disease in a living being, as well as to a method for the treatment of a tumor disease or/and of an infection in a living being.

2. Related Prior Art

Therapeutic and diagnostic substances which are used in the therapy and diagnosis of tumor diseases or infections, are generally known in the art.

A therapeutic approach in the treatment of tumor and infectious diseases relates to the administration of drugs which cause a damage, necrosis, or growth inhibition of the tumor cells or infected cells.

The so-called cytostatics constitute a group of mostly synthetically produced and chemical heterogeneous substances which have toxic effects on different biological cells, and inhibit cell growth and cell division.

The cytostatic or cytotoxic substances, respectively, which are available so far, do not have a selective effect on tumor cells but harm normal tissue as well. Especially affected are tissues with high cell division rates, as, for example, gonads, hair follicles, and cells of the blood-forming system. An overview about the development of cytostatics is given in S. N. Gardner and M. Fernandes (2004), “Cytostatic Anticancer Drug Development”, J. Exp. Ther. Oncol., pages 9 to 18.

Improvements in the treatment and diagnosis of tumor and infectious diseases were made after the discovery of antigens which are expressed on the surface of infected or transformed cells. Such surface proteins on tumor cells are referred to as so-called tumor antigens. Based on these findings, there are efforts to develop substances which specifically recognize these tumor antigens and thereupon mediate a selective attack on the tumor cell. This is for example attempted by means of antibodies specific for these tumor antigens, which are coupled to cytotoxic substances. Another corresponding approach relates to a specific stimulation of the immune system against tumor cells by administering these tumor antigens which can be modified, or by the direct application of so-called tumor vaccines containing these tumor antigens. An overview about this therapeutic approach is given in Joseph N. Blattmann and Philip D. Greenberg (2004), “Cancer Immunotherapy: A Treatment for the Masses”, Science, Vol. 305, pages 200 to 205.

However, a disadvantage of this approach is that by most of the currently known tumor antigens malignant cells cannot be distinguished from benign neoplasms or even from normal cells, so that a targeted attack on malignant cells is not possible with such antigens or will not give satisfactory results. Furthermore, there are infected and transformed cells described in the art, which show no special immunogenicity at all. In this case, a distinction between these cells and normal cells and, therefore, a targeted therapeutic intervention by the means of surface markers is not possible.

It is also known in the art that in tumor cells regulatory mechanisms are altered when compared with normal cells. The reason for this could be a genetic alteration of signal transduction factors. A summary of genetic alterations in tumor cells can be found in Douglas Hanahan and Robert A. Weinberg (2000), “The Hallmarks of Cancer”, Cell, Vol. 100, pages 57 to 70.

Among experts it is known that in certain tumor cells permanent or increased growth signals of structurally intact but amplified surface receptor kinases are transduced into the cell, whereas in normal cells growth impulses are only induced at specific times. Equally, a huge number of tumors have been described to show activating mutations of intracellular factors of the signal transduction cascade, such as for example mutations in the ras protein, a monomeric GTPase having proliferation regulating activity. The ras protein is mutated in 30% of human tumors. This mutation that is mainly described for exocrine pancreas carcinoma and in colon carcinoma, causes the loss of the hydrolytic activity of the ras protein resulting in a permanent active and proliferation-stimulating form of this protein. Also observed in tumor cells is the inhibition or knockout of growth inhibitory factors like the retinoblastoma (Rb) or the p53 protein, the so-called tumor suppressors. Also described in the art is an alteration of the telomerase activity in tumor cells which is connected with the acquisition of immortalizing properties. These cells have the property that they, unlike normal cells, can be permanently cultivated in cell culture. Further summarizing reports thereto can be found in William C. Hahn and Robert A. Weinberg (2002), “Rules for Making Human Tumor Cells”, N. Engl. J. Med., Vol. 347, No. 20, pages 1593 to 1603; or in William C. Hahn and Robert A. Weinberg (2002), “Modelling the Molecular Circuitry of Cancer”, Nat. Rev. Cancer, Vol. 2(5), pages 331 to 341.

Irish et al. (2004), “Single Cell profiling of Potentiated Phospho-Protein Networks in Cancer Cells”, Cell, Vol. 118, pages 217 to 228, have discovered that several transduction mechanisms which are controlled by the phosphorylation of signal molecules are altered in tumor cells. On account of these findings, the authors drew up tumor-specific multidimensional molecular phospho profiles. However they do not describe in detail how exactly the signal transduction factors in tumor cells are altered in comparison to those in non-tumor cells. Further there is no description about the relation between the altered signal molecules and the cell cycle, since the experiments described in this document were only performed over a very short time period.

Despite of these discoveries regarding altered signal transduction mechanisms in tumor cells, the experts have so far failed in providing a substance that therapeutically or/and diagnostically benefits from these alterations.

### SUMMARY OF THE INVENTION

Therefore, it is an object of the present invention to provide a substance which recognizes and combats transformed or/and infected biological cells in a specifically targeted manner, and which does not show the disadvantages of known substances.

This object is achieved by providing a therapeutic or/and diagnostic sub-stance that indirectly or directly reacts with at least two molecules which largely simultaneously appear exclusively in a transformed or/and infected biological cell, said reaction resulting in the induction of a biological or/and detectable property.

According to the invention, a substance is understood to be both, a purely chemically defined substance, like an organic or inorganic compound, as well as a biological substance, like a peptide or a protein or an RNA/DNA aptamere. Therefore, a substance can be a low molecular agent, a so-called “small molecule” as well as a viral or molecularly modified particle or an antibody.

A therapeutic or diagnostic substance refers to a kind of substance that is designated for use in therapeutic or diagnostic applications.

According to the invention, a transformed cell refers to a kind of cell that has undergone a malignant, neoplastic or oncogenic transformation, i.e. a cell that has under-gone an alteration resulting in an altered growth behavior. Causes for such alterations can be chemical or physical noxa as well as an infection by oncogenic viruses. Also spontaneous mutations are observed which lead to a transformation of the affected cell. Frequently, transformed cells acquire the ability to form tumors.

According to the invention, an infected cell refers to a kind of cell which has been penetrated by pathogens, like for example by viruses, bacteria, fungi or microorganisms of all kinds, or parts thereof, which have caused an alteration in the cell. In connection with this invention, this particularly refers to an infection of cells by oncogenic viruses, for example by so-called tumor viruses, such as certain adenoviruses, papilloma viruses, or herpes viruses, such as the Epstein-Barr virus (EBV). It has for example been shown for EBV that after an infection, homologs of kinases are expressed in the cell, which interfere with the regulation of the signal transduction. Infective pathogens also include representatives of the so-called RNA tumor viruses or retroviruses as well as of organisms in general, which interfere with and alter the signal transduction mechanisms of the infected cell.

The at least two molecules with which the substance according to the invention reacts, refer to cell-owned compounds such as for example enzymes, which differ from each other in their activity or/and specificity or/and affinity for or/and accessibility to reactants or in other characteristics. According to findings of the inventors, these molecules do not appear simultaneously in normal, i.e. in non-transformed or non-infected cells. These differences in the chronological order of appearance of the two molecules, which can be observed in single normal cells, can, for example, be the result of cell cycle-specific regulatory mechanisms. It is, in fact, known that for example cyclin-dependent kinases (CDK) are regulated both in their activity as well as in their availability over the cell cycle, so that these proteins only appear at specific times in the cell cycle. The phenomenon of the non-simultaneous appearance of the molecules in question in single normal cells can be traced back to other regulatory intra- and extra-cellular phenomena, such as for example time-coordinated mitogenic impulses.

According to the invention, non-simultaneous appearance of the two molecules means that these two molecules either are not present at the same time in one normal single cell, or are not active at the same time, or do not display their activity at the same time or in the same manner. That is to say, that simultaneously, on the contrary, means that in a transformed or infected cell these two molecules are present or active in one single cell essentially at the same time, i.e. over longer times within the cell cycle or in the arrested state of the cell (G0 phase) and not just punctiform. This concurrence in transformed or/and infected cells means, according to the invention, that the two molecules appear essentially simultaneously in single cells.

The inventors have selected by way of example two molecules involved in healthy cells in the regulation of the cell cycle at different points in time, i.e. MAP kinase (ERK) and the retinoblastoma protein (Rb) or cyclin-dependent kinases (CDK), respectively. Further such two molecules can be chosen by the skilled person. Numerous literature articles provide the identity and activity kinetics of molecules. Examples of such molecules which are involved in the regulation of the cell cycle are provided by Cho et al. (2001), “Transcriptional Regulation and Functioning During the Human Cell Cycle”, Nature Genetics, Vol. 27, pages 48-54, especially on page 51, FIG. 2, and by Douglas Hanahan and Robert A. Weinberg (2000; l.c.), especially on page 59, FIG. 2, as well as by William C. Hahn and Robert A. Weinberg (2002; l.c.), especially on page 337, FIG. 2. These typical diagrams depict the identity of molecules that are involved in the regulation of the cell cycle as well as their successive order of activity. With such information, one can readily select two other molecules involved in the regulation of the cell cycle, which two molecules are active at different points during the cycle. Thus, the skilled artisan is apprised of potential two molecules as a reaction partner for the substance according to the invention.

The reaction of the substance with the at least two molecules can take place in a direct way, i.e. via direct steric interaction of the substance with the two molecules, as well as indirectly, for example via interposed factors or interposed molecules.

The reaction of the substance according to the invention with the two molecules can result, for example, in an addition or separation of molecules or parts of molecules, such as phosphate groups, to and from the substance, or to and from interposed factors, or in a rearrangement of groups or parts of the substance or of interposed factors.

A biological property refers to a property which is specifically induced by the substance due to the reaction with the at least two molecules, and, which for example, presents itself as a biological activity in the cell. In general, said property might refer to an enzymatic, chemical, biochemical or physical activity which is induced by the substance.

According to the invention, a detectable property refers to a property which is induced by the reaction of the substance with the at least two molecules, and presents itself as a measurable value that indirectly or directly emanates from the so-reacted substance. As a detectable property, every measurable property can be considered, e.g. an activity that can be detected by means of chemical, biochemical or physical methods known in the art.

The biological or/and detectable property can be induced as a result of the metabolization of substance in the cell, as a result of the transformation of the substance into a different state of activity or a different structure, or because of the expression of a product produced by the cell, such as an enzyme, or because of the modification of the activity of a cell-owned protein, whereby all this results from the reaction of the substance with the at least two molecules.

Said metabolic or expressed product resulting from the induction of the biological or/and detectable property, or even the reacted substance itself can directly act as a therapeutic or diagnostic agent, can cause an indirect reaction, as for example an immunoreaction, or can act as a mediator by enabling a targeted attack of a therapeutic or diagnostic agent.

According to the invention, due to an appropriate construction of the substance which is up to the discretion of the skilled person, the biological or detectable property that is explained above in more detail, is only induced if said substance reacts with the at least two molecules in an essentially simultaneous manner. A reaction of the substance according to the invention, with only one of the two molecules does not result in the induction of the biological or/and detectable property. Thus, the said substance may e.g. function as a pro-pro-drug requiring not only one but essentially two modifications simultaneously in the same transformed or infected cell to become active.

The object underlying the invention is herewith totally achieved.

The inventors have demonstrated for the first time on a single cell level, that intracellular molecules, such as for example factors of the signal transduction cascade, appear essentially simultaneously in a transformed or an infected biological cell. Said molecules are, for example, simultaneously active in the cell cycle over a longer time, whereas such molecules in a normal, i.e. healthy cell appear in a clearly distinguishable chronological order, e.g., are active in the cell cycle at different times.

This phenomenon of the concurrence of the appearance of molecules exclusively in transformed or infected cells, that has been discovered by the inventors is not described in the art. It is known that in larger populations of tumor cells, as for example in cell cultures, tissue structures or whole organs, certain signal molecules are constitutively active. For example, in 30% of all tumors the ras kinase is permanently sending signals into the cell. Therefore, when examining transformed cells on multi cell level, a parallel appearance of the ras kinase and other signal molecules, such as CDKs, can be assumed, even if this takes place just in a punctiform manner within the cell cycle. This assumption in the art regarding cell populations does not allow any conclusions to the conditions in a single cell. This has so far prevented the concept of a targeted substance that is effective in each single transformed or/and infected cell.

On the basis of these new findings obtained by the inventors it is now possible for the first time to design the substance according to the invention, which induces a biological or/and diagnostic property in single transformed and infected cells, due to an essentially simultaneous reaction of said substance with the two molecules.

This property is not induced in normal, e.g. healthy cells, since no corresponding reaction is taking place, because of the clearly distinguished chronological order of appearance of the at least two molecules in said cells. Instead of this, in normal cells only a reaction of the substance, according to the invention, with one of the two molecules takes place since the respective other molecule is not active, present or accessible at the same time, for example due to cell cycle-specific regulations.

The substance can be designed in such a manner, that a reaction of the substance with only one molecule, or a reaction first with one of the two molecules and after a sufficient time period with the respective other molecule, or that no reaction at all results in an instability, a direct or indirect degradation, an inactivation, the discharging or any other inoperativeness of the substance according to the invention. An induction of the biological or/and detectable property in normal or healthy cells does, therefore, not take place.

The inventors have therefore provided a substance that induces effects in transformed and infected cells in a highly selective and specific manner, whereas these effects are essentially not induced in normal cells. Thus, the substance represents a valuable tool in the therapy and in the diagnosis of tumor and infectious diseases.

The substance according to the invention is preferably constructed in such a manner, that it reacts with two cellular enzymes, especially with two kinases, which are involved in the regulation of the cell cycle.

The afore-mentioned measure has the advantage that key factors of the regulation of the cell cycle are utilized in order to induce the biological or/and detectable property. The inventors have found that, for example, two enzymes, preferably two kinases or phosphotransferases, are essentially simultaneously active in a transformed or infected cell with no simultaneous action being observed in a healthy cell.

The substance according to the invention is designed in such a way, that it induces the biological or/and detectable property in the cell after the reaction with the two enzymes or two kinases. Such a design which is up to the discretion of a man of the art, is especially useful, since it provides a therapeutic or diagnostic tool that is highly selective for a transformed or infected cell. With this measure, a biological property can also be induced in a cell that has been infected by viruses which are described in the art and which cause an activation of cell cycle-regulating kinases. According to the invention, this also includes kinases which are present in cell cycle-arrested cells, i.e. in such cells which are resting in G0 phase.

According to a preferred further embodiment, the substance according to the invention is designed in such a manner, that the biological or/and detectable property is induced in a case where one of the two molecules is an enzyme of the ras/raf signal transduction cascade, and the other of the two molecules is an enzyme of the CDK signal transduction cascade e.g. of the CDK2, CDK4 and/or CDK6 signal transduction cascade.

The ras/raf signal transduction pathway results in an activation of the MAP kinase (mitogen-activated protein kinase, also called ERK1) via a cascade of, essentially, phosphorylation events; cf. William C. Hahn and Robert A. Weinberg (2002, l.c.). The CDK signal transduction pathway, e.g. the CDK2 signal transduction pathway, results in an activation of the cyclin-dependent kinases, e.g. of the cyclin-dependent kinase 2, via the stimulation of the transcription or/and the activation of the molecule by phosphorylation or association with binding partners, e.g. cyclins. According to the invention, the CDK can consist of any catalytic cdk Subunit, e.g. cdk1, cdk2, cdk4 or cdk6 and any of the regulatory subunits cyclin A, E, D or others. Thus, any active CDK is considered; cf. for this A. W. Murray (2004), “Recycling the Cell Cycle: Cyclins Revisited”, Cell, Vol. 116 (2), pages 221 to 234. The content of this publication is incorporated herein by reference.

This preferred afore-mentioned measure is advantageous, since it therapeutically and diagnostically utilizes a phenomenon which has been detected by the inventors for the first time as exclusively appearing in transformed and infected cells. Gong et al (1994), (“Unscheduled Expression of Cyclin B1 and Cyclin E in Several Leukemic and Solid Tumor Cell Lines”; Cancer Research 1994, 54:4285-8) describe the “unscheduled” expression of cyclins in malignant cells. They did not, however, analyze CDK activity. Expression of a discrete subset of cyclin is not equivalent of CDK activity, since CDK activity is also determined by the catalytic cdk subunit and by posttranslational modifications. It is in fact assumed in the art, that the ras kinase is constitutively active in cultures of tumor cells or other tumor cell populations examined in total, but the kinetics of the kinase activity on a single cell level is, up to now, totally unclear. However, it has now been shown for the first time on a single cell level, that in transformed and infected cells, both the ras/raf as well as the CDK signal transduction cascade is largely proceeding simultaneously over longer times, resulting in the simultaneous appearance of the activities of the single factors of the corresponding two signal cascades, such as of, for example, the MAP kinase and the CDK kinase, in transformed or infected cells.

This discovery was especially surprising, since in normal, i.e. healthy cells, both signal transduction cascades proceed in a sequential manner. In normal cells the CDK2 complexed with cyclinE or A are usually active in the late G1 phase or at the beginning of the S phase in which the DNA replication takes place. On the other hand, in normal cells the MAP kinase is usually active very early in the G1 phase, but no longer at the beginning or during the S phase. As a result of this, as the inventors have shown for the first time, in a normal human dividing cell there is a time difference between the activity peak of CDK, especially of CDK2, and the activity peak of MAP kinase, which is about 24 hours.

The simultaneous progression of signal transduction pathways, that is observed in the single transformed and infected cell for a longer time over the cell cycle, with these signal transduction cascades being mutually exclusive in normal cells, has been discovered by the inventors for the first time. The feat of the inventors is that they have made therapeutic use of their observation. In this connection, the inventors have realized that the selectivity of the substance for transformed and infected cells is especially pronounced, if said substance is designed in such a way, that it can react, on the one hand with any enzyme of the ras/raf signal transduction cascade, and on the other hand with any enzyme of the CDK signal transduction cascade, especially of the CDK2 signal transduction cascade, so that therewith a biological or/and detectable property is induced.

The substance can also be designed in such a way, that the property in question is induced, when a reaction with enzymes occurs, which are involved in at least two other such signal transduction cascades which do not proceed simultaneously in normal or healthy cells. Examples of such enzymes can be found in the online Atlas of Genetics and Cytogenetics in Oncology and Haematology (available on the website of Infobiogen) and the National Center for Biotechnology Information Online Mendelian Inheritance in Man database (available on the website of the National Center for Biotechnology Information), the content of which is incorporated into the present application by reference. Further examples are given in the article of Hahn and Weinberg (2002, l.c.).

According to a preferred embodiment, the substance according to the invention is a substrate for the at least two molecules, i.e. the substrate can be referred to as a double pro-drug or a pro-pro-drug.

This measure has the advantage that therewith a substance is provided that reacts directly with the two molecules in the envisaged manner, without the need of considering interposed factors. The substance can preferably comprise two different phosphorylation sites, one of the two being, e.g., specifically recognized and phosphorylated by the MAP kinase, and the other one being, e.g., specifically recognized and phosphorylated by a CDK kinase, e.g. the CDK2 kinase. Only the largely simultaneous reaction of the substrate, i.e. the phosphorylation of both the CDK phosphorylation site, especially the CDK2 phosphorylation site, and the MAP kinase phosphorylation site at largely the same time results in the induction of the biological or/and detectable property.

The substance also can be designed in a different way so that it represents a substrate that is, in the case of a largely simultaneous reaction with the two molecules, directly converted into its active form, e.g. due to the establishment of accessibility to active centers or reactive groups of the substance, which have been sterically inaccessible or chemically inactive before the reaction with the two molecules.

Such a design of the substance according to the invention, as a substrate for the two molecules (pro-pro-drug) can be managed by a skilled person without undue efforts. For example, the CDK and MAP kinase phosphorylation sites which differ from each other, are well known in the art: the CDK2 phosphorylation site is, for example, described in the publication of Brown et al. (1999), “The Structural Basis for Specificity of Substrate and Recruitment Peptides for Cyclin-dependent kinases”, Nat. Cell. Biol., Vol. 1(7), pages 438 to 443, and of Songyang et al. (1994), “Use of an Oriented Peptide Library to Determine the Optimal Substrates of Protein Kinases”; Curr. Biol., Vol. 4(11), pages 973 to 982. The phosphorylation site for the MAP kinase is, for example, described in the publication of Songyang et al. (1996), “A Structural Basis for Substrate Specificities of protein Ser/Thr Kinases: Primary Sequence Preference of Casein Kinases I and II, NIMA, Phosphorylase Kinase, Calmodulin-dependent Kinase II, CDK5, and Erk1”, Mol. Cell. Biol., Vol. 16(11), pages 6486 to 6493. The phosphorylation sites can be prepared by means of commonly used methods of peptide synthesis.

According to a preferred embodiment the substance according to the invention, is designed in such a way, that when reacting with at least one cellular factor besides the two molecules, the induction of the biological or/and detectable property is modified.

According to the invention, a cellular factor refers to any intracellular molecule such as for example a protein with a defined activity, that indirectly or directly interacts with the substance, and by doing so, modifies the induction of the biological or/and detectable property. A modification could mean, that the induction of the property as a result of the interaction with the cellular factor, is enhanced or even actually takes place. A modification can also mean that the induction of the property resulting from the reaction with the cellular factors, is reduced or does not take place at all.

This measure has the advantage that herewith the induction of the biological or/and detectable property is even better controlled. Furthermore, the substance can be designed in such a way, that it does only react with such a cellular factor that is present in a transformed or infected cell, and that the induction of the property is, due to this reaction, enhanced. On the contrary, it is also possible to design a substance in such a way, that a reaction only takes place with such cellular factors which are present in a normal or healthy cell. By the latter measure, the substance according to the invention could be altered in such a way, that an induction of the biological or/and detectable property, which might occur due to unforeseen events even without the presence of the two molecules, is prevented. The therapeutical or diagnostic utility is further increased by this security measure.

According to the invention, it is further preferred if the cellular factor consists of an apoptotic or anti-apoptotic molecule or of the telomerase enzyme.

This measure provides an improvement of the selectivity and specificity of the substance according to the invention, for transformed or/and infected cells. It is known, that the telomerase is especially active in transformed cells, whereas no or merely weak telomerase activity can be detected in healthy cells. Comparable conditions apply to anti-apoptotic molecules. These are essentially active in transformed cells, but are not active or merely in much less degree in healthy cells. By an appropriate design of the substance according to the invention, the latter is increased in its ability to induce a biological or/and detectable property by the interaction with the telomerase or with anti-apoptotic molecules.

In transformed or in infected cells, pro-apoptotic mechanisms are very often inactivated. This occurs, inter alia, by the inactivation or the degradation of pro-apoptotic molecules which are mainly present or active in normal cells. By an appropriate design of the substance according to the invention, the latter will be altered after a reaction with pro-apoptotic molecules in that way, so that the induction of the biological or/and detectable property is no longer possible.

It is preferred if the substance according to the invention is designed in such a way, that its entrance or uptake into the cell or/and cellular compartments is enabled.

By this measure, it is assured that the substance in fact can induce the biological or/and detectable property in the interior of the cell or in the envisaged cellular compartments, such as for example the cytoplasma, or the nucleus. Such a design can be realized by providing a segment of the molecule, that mediates the permeability of the latter through the membrane, or by another segment that enables the passive or active transport of the substance into the cell.

This can also occur by providing an area or a segment in the molecule, which establishes the affinity and internalization of the substance for or into the cell, respectively, such as for example by means of an antibody that might be modified, or by an aptamer or another ligand, which are bound to the substance. By this measure, the selectivity of the substance is further increased. For example, ligands can be provided, which enable the entrance or uptake into very specific transformed or infected cells, such as cells of a particular tumor or cells which were infected by a particular pathogen. Therefor cell type-specific surface markers, for example tumor antigens, can be used to which ligands which are provided at the substrate according to the invention, bind in a selective manner. According to this preferred variation, the design of the substance can be realized by the packaging of the substance into a transport vesicle.

According to a preferred embodiment, the substance according to the invention is designed as a low-molecular weight active agent.

Low-molecular weight active agents are also referred to as “small molecules”. This is a generic term for chemical substances having activities in biological systems. The molecular weights of these compounds are usually below about 1000 to 1200 Dalton, in some cases they can also be above that weight. The advantage of this measure is, that herewith the substance can be produced on a large scale by means of well established synthetic methods, and that the substance is sufficiently stable. Furthermore, chemically defined or biological matrices which are known in the art such as peptides can be used, and their properties can be optimized by chemical synthesis by using the so-called “rational drug design”, which is also referred to as “molecular evolution” or “specificity evolution”; cf. Böhm et al. (2002), “Wirkstoffdesign”, Spektrum Akademischer Verlag, Heidelberg. New developments in the field of the production of low molecular weight active agents have been summarized by Nature magazine (available on the website of Nature under the terms “horizon”, “chemicalspace”, and “highlights”), the content of which is incorporated into this application by reference.

It is preferred if the substance according to the invention is a peptide.

This measure is advantageous since in this case the substance can be produced in an easy manner by means of well known methods of peptide chemistry. The substance can be designed as a substrate for the two molecules in an easy way, for example by providing a segment comprising a phosphorylation site for the CDK2 kinase, and a segment comprising a phosphorylation site for the MAP kinase. Moreover, in this embodiment peptide segments can easily be provided, which confer upon the substance its permeability through the cellular membrane. These kinds of peptide segments are well known in the art, and consist preferably of a sequence of arginine residues.

Another advantage of the design of the substance according to the invention as a peptide consists in the fact, that a peptide is a suitable template or matrix for the preparation of a “small molecule”. For example, a peptide can be synthesized having an affinity for the two molecules as well as the intended biological property, subsequently a co-crystal consisting of said peptide complexed with the two molecules can be obtained via standard methods. With the aid of said co-crystal, a corresponding low molecular substance can be derived by means of “molecular evolution” or “specificity evolution” in silico, which then, in turn, can be chemically synthesized on a large scale. This pathway which uses the peptide as a template for a corresponding “small molecule” and, therefore, as a kind of intermediate product of the substance according to the invention, is clearly predetermined for a skilled person.

An easy handling of the substance is also made possible by another embodiment according to the invention, by which segments are provided in the substance, which mediate a binding to an affinity column, as for example a segment comprising histidine residues, or a tag consisting of glutathion-S-transferase (GST). These segments or tags can be easily provided in a peptide. The separate functional segments can then, e.g., be connected to each other by connecting sequences, so-called “linkers”. By the design of the substance as a peptide, therefore, a flexible and easy preparation according to the intended property is made possible.

The before-mentioned easy preparation of such peptides by which a user-defined reaction can be induced in the cell via a reaction with cell-owned molecules, is described in the art. For example, Nguyen et al. (2004), “Caged Phosphopeptides Reveal a Temporal Role for 14-3-3 in G1 Arrest and S-phase checkpoint Function”, Nature Biotechnology, Vol. 22, pages 993 to 1000, describe the preparation of a peptide construct that can be introduced into biological cells, and that shows a reaction with cell cycle-regulating molecules after an activation by UV radiation. The data presented there further prove the enablement of the present invention.

Another advantage of the substance being designed as a peptide consists in the fact, that a peptide can be easily prepared, mutated or otherwise altered by means of molecular biological methods, for example by using an expression vector in prokaryotic or eukaryotic expression systems. Therefore, a further subject of the invention also relates to an expression vector that encodes a corresponding peptide according to the invention. It goes without saying, that the expression vector according to the invention, can also comprise segments which enable or promote the expression in a cell type-specific manner, such as promoters, enhancers etc., or segments which enable a handling of the vector in the laboratory, such as for example segments which encode resistances against antibiotics, cleavage sites for restriction enzymes, polylinkers, etc.

It is also conceivable that the expression vector according to the invention, is directly used as a therapeutic or/and diagnostic substance. By means of an appropriate design, the expression vector is introducible into biological cells within which it is expressed. This design also has the advantage that nucleic acids are much more stable and robust compared to proteins and can be stored for an almost unlimited time. The expression vectors according to the invention are produced by methods described in the art. As an example for a corresponding manual the treatise of Joseph Sambrook and David W. Russel (2001), “Molecular Cloning—A Laboratory Handbook”, Cold Spring Harbor Laboratory Press, Second Edition, can be cited, the content of which is herewith incorporated into the present application by reference.

Preferably, the substance is designed in such a way, that the biological property has an either direct or indirect toxic effect on the transformed or/and infected cell.

By this measure, a substance is created which is selectively toxic for transformed or infected cells only, whereas it is largely safe for normal cells, since only the largely simultaneous reaction with the two molecules causes the induction of the toxicity, whereas a reaction with only one or none of the two molecules includes no or merely a negligible toxic activity (pro-pro-drug).

A toxic property refers to such an activity which has a direct or indirect lethal effect on transformed or infected cells, for example by inducing apoptosis, necrosis, or oncosis, by inhibiting the metabolism, the signal transduction, the proteasom or the transcription activity interaction with the spindle apparatus of the cell, etc. A toxic property also refers to an activity which causes an arrest of the cell cycle or results in a well-aimed activation of the immune system or the expression of an antigenic determinant, resulting in an attack on the transformed or infected cell.

The phosphorylation sites for the two molecules are preferably provided within the sequence of the p53 molecule or segments thereof.

By this measure it is meant that the sequence of the p53 molecule, preferably of the human variant, or one or several parts thereof, are a part of the substance according to the invention. This part comprises the phosphorylation sites for the two molecules, for example the two kinases which appear in transformed or infected cells in a largely simultaneous manner. Therefore, the phosphorylation sites can, for example, be embedded into the sequence of the p53 molecule, or can be provided by the phosphorylation sites which naturally are located within the p53 molecule, or can replace the natural phosphorylation sites of the p53 molecule.

The p53 molecule is a tumor suppressor protein that is regulated in its activity by phosphorylation events. The phosphorylation of the p53 protein causes an increase of its stability. In this case, the p53 protein acts as an active transcription factor and causes an activation of cell cycle-arresting proteins, such as p21^{Cip1}, or the beginning of apoptosis.

With the provision of the phosphorylation sites for both kinases in the p53 molecules or functional segments thereof, for example of phosphorylation sites for the MAP and CDK2 kinase, a tool is created that displays p53-specific activities. For this, the substance is designed in such a way, that only in the case of a phosphorylation of both phosphorylation sites or of a phosphorylation of both phosphorylation sites at largely the same time, p53-specific activities are displayed, so that only in transformed or infected cells an arrest of the cell cycle or the beginning of apoptosis is induced.

According to a further embodiment, the substance is designed in such a way, that the detectable property is detectable by means of imaging methods.

This can, for example, be realized by designing the substance as a photoactivating molecule that emits a detectable signal after a reaction with the two molecules. The reaction of the substance according to the invention with the two molecules can also cause an activation of a further molecule which then emits a detectable property.

A suitable detectable property refers, for example, to luminescence or fluorescence, phosphorescence, bioluminescence, radioactivity or any other detectable signal. It is also, for example, possible to detect the reaction product that was generated by the reaction of the substance according to the invention with the two molecules, by the usage of antibodies or other ligands in a direct or indirect manner.

Within the frame of imaging procedures, methods such as tomography, FACS (fluorescence activated cell sorting), FRET (fluorescence resonance energy transfer), fluorescence microscopy, immunoblotting, ELISA, radiological methods, etc. can be used.

Another subject of the present invention relates to a composition, preferably to a pharmaceutical composition, that exclusively induces a biological or/and detectable property in a transformed or/and infected cell.

In the context of the invention, a property that is exclusively induced in transformed or/and infected cells refers to an induction that is at least largely if not totally avoided in normal cells, or an induction that can be tolerated in normal cells when considering the therapeutical or diagnostic benefits.

With their data the inventors give evidence for the first time, that a targeted and selective attack on transformed or infected biological cells is possible, whereas normal or healthy cells are almost completely unaffected. Furthermore, the inventors provide a substance or a composition, respectively, for the first time, that induces a biological or/and diagnostic property exclusively in transformed or/and infected cells in a highly selective manner. This has not been achieved in the art so far.

The composition preferably comprises the substance according to the invention, or the expression vector according to the invention, and, if appropriate, a pharmaceutical acceptable carrier. The production of such a pharmaceutical composition is well described in the art. In this connection the publication of Arthur A. Kibbe (2000), “Handbook of Pharmaceutical Excipients”, American Pharmaceutical Association and Pharmaceutical Press, Third Edition, can be cited, the content of which is incorporated into the present application by reference. The choice of the appropriate concentration of the substance in the composition is up to the discretion of the skilled person and can be determined by means of simple experiments, for example by titration experiments. In most of the cases it is also necessary to determine the optimum concentration of the active agent individually, depending on the patient to be treated.

The composition according to the invention, preferably comprises activity enhancing agents. This includes all compounds which increase the induction of the biological or/and detectable property by the substance according to the invention. Appropriate activity enhancing agents for an application in vitro are tumor promoters, such as phorbole-12-myristate-13-acetate (PMA) or ionomycin. For an application in vivo cytostatics, antibodies such as herceptin or rituximab, or growth factors, such as G-CSF or FGF can be used. These activity enhancing agents are to be used in an appropriate concentration, so that the simultaneous appearance or activity of the at least two molecules is enhanced exclusively in the transformed or infected cells, whereas normal or healthy cells are not affected. Further appropriate activity enhancing agents used in the pharmaceutical substance according to the invention are, when a therapeutic application is intended, cytostatics which are well known in the art, or other active agents which are used in the therapy of cancer diseases or infections. Also, so-called “sensibilizers”, as for example bispecific antibodies, are possible activity enhancing agents.

Against this background, the substance according to the invention, or the expression vector according to the invention, can be used for the preparation of a pharmaceutical composition for the treatment of transformed or/and infected biological cells.

A further subject of the invention relates to a method for diagnosing a tumor disease or/and infection in a living being, comprising the following steps: (a) providing a biological sample to be analyzed; (b) analyzing the appearance of molecules in single cells of the biological sample, and (c) correlating the finding of essentially simultaneously appearing molecules in single cells of the sample, which exclusively appear essentially simultaneously in a transformed or/and infected biological cell, with a positive diagnosis.

According to the invention, a biological sample encompasses isolated cells and tissues as well as whole organisms, e.g. a human or animal being. In the latter case the tissue or organism has to be treated in order to provide single cells. It is a matter of routine skill in the art that any multi-cellular tissue can be disaggregated by an enzymatic treatment, followed by wash and centrifugation steps, causing the digestion of the extra-cellular matrix and isolation of the single cells constituting the actual tissue; see e.g. Salih et al. (2000), “Constitutive Expression of Functional 4-1BB (CD137) Ligand on Carcinoma Cells”, J. Immunology 165, pages 2903-2910; the content of which is incorporated herein by reference.

The diagnostic method can be performed with isolated biological material in the laboratory, which means in vitro, but also with a living organism, i.e. in vivo or in situ.

While the applicants have selected by way of example two molecules involved in healthy cells in the regulation of the cell cycle at different points in time, i.e. MAP kinase (ERK) and Rb or CDK, respectively, the analysis of other appropriate molecules is readily permitted because the identity and cell-specific activity of other such molecules is well-known in the prior art; see e.g. Douglas Hanahan and Robert A. Weinberg (2000; l.c., especially page 59, FIG. 2), and William C. Hahn and Robert A. Weinberg (2002; l.c., especially page 337, FIG. 2). These typical diagrams depict the identity of molecules that are involved in the regulation of the cell cycle as well as their successive order of activity. With such information, the skilled artisan can readily select two other molecules involved in the regulation of the cell cycle, which two molecules are active at different points during the cell cycle. If essentially simultaneous activity of those other two molecules is detected, i.e. the orderly and timely progression of the cell cycle has been disrupted, it can be concluded, according to the teachings of the present invention, that the cells of the analyzed sample are transformed and a malignancy is thus diagnosed.

It is further within the knowledge of the skilled artisan to judiciously select the molecule pairs to be analyzed, further reducing the level of experimentation required. Depending on the particular tumor disease of interest, the art contains numerous examples of molecules which are involved in the formation of a specific tumor; see e.g. Vogelstein et al., “Cancer Genes and the Pathways They Control”, Nature Medicine, Vol. 10, No. 8, pages 789-799, especially page 791, table 1; the content of which is incorporated herein by reference. For example, to develop a diagnostic method to detect a colon cancer, the skilled artisan can easily locate the published and well-described finding of the adenoma-carcinoma sequence that describes a multi-step development of a colon tumor with sequential mutations of APC, ras, and p53. Consequently, the skilled artisan could chose a kinase molecule down-stream of APC (a molecule of the WNT signaling module as an early or G1 event) or a molecule downstream of ras (also early G1), and examine the occurrence of significant kinase activity of later signaling events like CDKs or molecules of the p53 pathway. Alternatively, the skilled artisan might chose other molecules of later pathways such as, for example, molecules involved in the separation of chromosomes. In the case of essentially simultaneous significant activity of the two (i.e., early and late) pathways, a colon tumor could be diagnosed.

The analysis and detection of molecules other than the MAP kinase and the Rb used in the embodiments requires little experimentation. While the inventors selected antibodies directed to the active, i.e. phosphor-related forms of these molecules, antibodies are commercially available which are directed to the active forms of any molecules involved in the regulation of the cell cycle.

A main advantage of this method is that, after a positive diagnosis has been made, information is obtained about which molecules in the transformed or infected cells do appear simultaneously compared to normal cells. This enables the physician in charge to apply therapeutic agents, even ordinary cytostatics, which specifically interact with the two molecules or interfere in the corresponding signal transduction pathways.

The analysis in step (b) is preferably performed by means of the so-called single cell profiling or FRET technology.

The method of single cell profiling that is for example described in Irish et al. (2004, l.c.), enables the analysis of intracellular events on a single cell level, such as the observation of molecules which appear simultaneously in the single cell, and activities of said molecules. Hereby for example the activity of enzymes or kinases can be measured in a single cell. By the single cell profiling method the formation of artifacts is avoided, which are induced during the analysis of cell cultures due to the methods used therein, for example by synchronizing the cells within the cell cycle. The cells to be analyzed can rather be analyzed against the background of their natural physiological cell cycle on a single cell level.

Within step (a) preferably a stimulation of the biological sample occurs by means of a tumor promoter, preferably by means of phorbole-12-myristate-13-acetate (PMA), ionomycin, a cytostatic, or an antibody, preferably herceptin or rituximab, or a growth factor, preferably G-CSF or FGF.

The inventors have realized that after a stimulation of the cells in vitro with such a tumor promoter, or in vivo with growth factors or cytostatics, a differentiation between transformed/infected biological cells and normal or healthy cells, is particularly easy. The substances herceptin and rituximab, for example, potentially activate the ras/raf pathway via Her2/neu or CD20, respectively, and therewith additionally increase the kinase activity in the transformed and infected cells. For healthy CD34 cells it has been found by the inventors, that after a corresponding stimulation the induction of kinase activities, for example of the MAP and CDK, especially CDK2 kinase activities, occurs in a distinctive chronological order, if compared to the induction of kinase activities in AML tumor cells, always on condition that single cells are analyzed. These differences which can be enhanced by such a stimulation, allow the diagnosis of a tumor disease or of an infection.

In the afore-described method preferably step (a) includes an incubation of a biological sample with the above-described substance according to the invention, or/and the above-described expression vector according to the invention. In this case step (b) includes the detection of the detectable property.

A further subject of the present invention relates to a method for the treatment of a tumor disease or/and infection in a living being, to which the above-explained substance according to the invention, or/and the above-explained expression vector according to the invention, is administered.

It goes without saying that the before-mentioned features and the features to be described below can be used not only in the combination indicated in each case, but also in different combinations or alone, without departing from the scope of the invention.

The subject-matters of the present invention are now explained by means of examples which are of purely illustrative character and do not limit the teaching according to the invention. Thereby, reference is made to the attached figures.

### BRIEF DESCRIPTION OF THE DRAWINGS

FIG. 1: Diagram of the progression of the ras/raf (pMAPK) and the CDK (pRb) signal pathways in transformed (AML) and healthy (CD34+) cells.

FIG. 2: FACS data demonstrating progression of the r as/raf (pMAPK) and the CDK (pRb) signal pathways in healthy hematopoietic stem cells (CD34+), cells from a patient with acute myeloic leukemia (AML), and a myelomonocytic leukemic cell line (HL60). Simultaneous activity of both, ras/raf and CDK pathways was exclusively detected in leukemic but not in healthy cells.

FIG. 3: Fluorescence imaging of the electrophoretic separation of substances specific phosphorylated by cyclin A/cdk2 kinase or ERK kinase in vitro.

FIG. 4: Fluorescence imaging of the electrophoretic separation of a diagnostic substance demonstrating different migration depending on phosphorylation status: single phosphorylation by either cyclin A/cdk2 kinase or ERK kinase (situation non-transformed cell): slow migration in PAGE; double phosphorylation by cyclin A/cdk2 kinase or ERK kinase (situation transformed cell): fast migration in PAGE.

FIG. 5: Kinetics of alterations in fluorescence demonstrating the induction of a detectable property in a diagnostic substance after a simultaneous phosphorylation by cyclin A/cd2: accelerated decrease of fluorescence.

### DESCRIPTION OF PREFERRED EMBODIMENTS

### Example 1

### Differential Signal Transduction in Normal/Healthy Cells and in Transformed Cells

CD34 positive blood stem cells were isolated by “magnetic cell sorting” (MACS). AML tumor cells were obtained from the peripheral blood of a patient suffering from acute myeloic leukemia (AML) M5 having >80% blasts, without any further manipulation. HL60 tumor cells were obtained internally at Eberhard-Karls-University, Tuebingen Germany.

10⁶ cells each were cultivated in 6-well plates. The cells were activated with PMA and ionomycin and, therewith, released into the cell cycle. Cells were fixed with 2% formaldehyde at different time points after incubation, and membranes were permeabilized with methanol.

Afterwards, the activities of the factors of the ras/raf signal pathway were analyzed via the phosphorylation state of the MAP kinase (pMAPK or pERK 1/2), and the activities of the factors of the CDK signal pathway were analyzed via the phosphorylation state of the retinoblastoma protein (pRB), a substrate of CDK.

For this the method of fluorescence activated cell sorting (FACS) was used, by means of which single cells can be analyzed. This method is described in detail in Irish et al. (2004, l.c.). For this method, the permeabilized cells were incubated with an rabbit anti-phospho-Rb, which specifically binds to phosphorylated Rb protein (pRb), followed by an PE-anti-rabbit antibody followed by an incubation with a FITC-conjugated anti-MAP-kinase antibody, which specifically binds to phosphorylated MAP kinase (pERK 1/2). The cells were analyzed in view the phosphorylation status of Rb and MAP kinase proteins at different time points after activation.

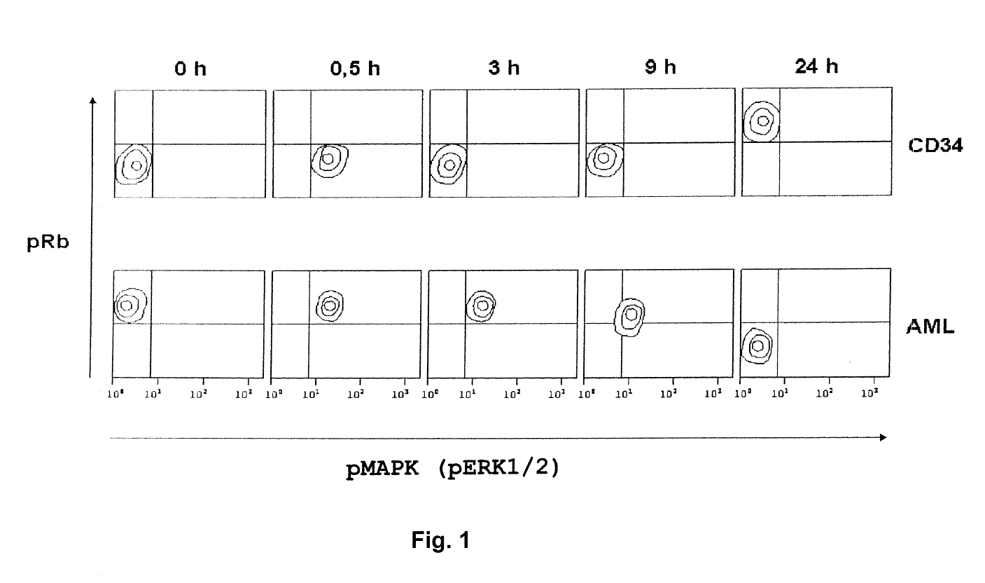

The results of this experiment are first schematically illustrated in the graph of FIG. 1. In this figure representative two-dimensional blots resulting from the FACS analysis are shown. The upper row depicts measurements on non-transformed CD34 positive cells at different time points as indicated. The lower row depicts corresponding measurements on transformed AML cells. On the x-axis the increasing phosphorylation of the MAP kinase is shown, whereas on the y-axis the increasing phosphorylation of Rb is shown. In order to simplify the orientation of the alterations of the signals, the blots have drawn-in intersecting lines.

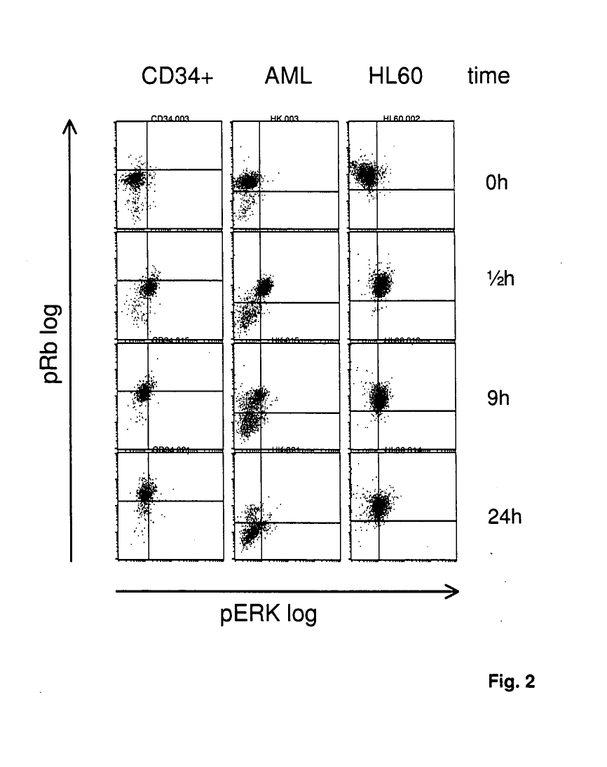

FIG. 2 shows the actual data measured in this experiment. In the left column the data measured on non-transformed CD34 positive cells are depicted, whereas in the middle and the right columns the corresponding data measured on AML and HL60 cells are depicted. Again, on the x-axis, the increasing phosphorylation of the MAP kinase (pERK) is shown in logarithmic units, whereas on the y-axis again the increasing phosphorylation of Rb (pRb) is shown in logarithmic units.

It can be seen from the blots, that in normal CD34 cells after 30 min, which could correspond to the early G1 phase, the MAP kinase (ERK) is present in its phosphorylated state. This is shown by a shift of the measured signal to the right. On the other hand, Rb is not in its phosphorylated state at that time, a shift of the measured signal into the upward direction did not occur.

In normal CD34 cells a phosphorylation of Rb did not take place until approximately 24 hours, which might correspond to the late G1 or early S phase where the MAP kinase is back in its non-phosphorylated state (FIG. 1, upper row; FIG. 2, left column). At even later measurements which are not shown in FIGS. 1 and 2, Rb is again in its non-phosphorylated state.

This observation in normal/healthy cells is in compliance with the knowledge in the art: in the early G1 phase the ras/raf pathway is activated as demonstrated by the phosphorylation and activation of the MAP kinase. In the late G1 or early S phase, respectively, the MAP kinase is inactive and therefore in its non-phosphorylated state. However, at this point in time the CDK signal pathway is activated, resulting in an active form of the CDK which phosphorylates different substrates, such as for example the RB protein (pRb). However, the MAP kinase and the CDK kinase are never simultaneously active.

The phenomena observed in the transformed cells were completely unexpected and are herein shown for the very first time: The kinetics of the activation of the ras/raf and CDK signal transduction cascades are strongly altered compared to the kinetics of the corresponding activation in normal cells. The Rb protein is found in its already phosphorylated state at the first measuring point (t=0 h). So it can be concluded, that the CDK is already in its active form. This is shown by an upward shift of the measured signal. Furthermore, the phosphorylated form of the Rb protein could be detected by radioimmunological methods or other means known in the art. Even 30 min after the activation, the MAP kinase also appears in its phosphorylated state simultaneously to pRb. This is shown by a shift of the measured signal to the right. This simultaneous phosphorylation of the Rb protein and the MAP kinase can be detected over long times during the measuring period. Regarding AML cells it is not until the 24 h measuring time point, that both the Rb protein as well as the MAP kinase are back in their non-phosphorylated states (FIG. 1, lower row; FIG. 2, middle column). In HL60 cells the Rb protein remains phosphorylated even 24 h after activation (FIG. 2, right column).

This difference between normal and transformed cells can also be observed without previous activation of the cells, in which case the simultaneous phosphorylation of the Rb protein and the MAP kinase in transformed cells is slightly less noticeable.

In parallel experiments the inventors were able to specifically inhibit the observed phosphorylation events of MAP kinase (ERK) and Rb proteins by the use of specific inhibitors, i.e. for inhibition of ERK protein phosphorylation the so-called MEK inhibitor (PD 98059) and for inhibition of Rb protein phosphorylation the so-called Rosco-vitine inhibitor were used. These experiments demonstrate the specificity of the observed phenomena.

Therefore, in the transformed cells one can surprisingly find an essentially simultaneous progression of both the ras/raf pathway as well as of the CDK pathway, even immediately after the release of the cells into the cell cycle. Active MAP kinase as well as active CDK can be detected essentially simultaneously in the transformed AML cells. The chronologically different appearances of the active MAP kinase (early measurement, 0.5 h) and the active CDK kinase (late measurement, 24 h) in the cell cycle, that can be observed in normal cells, is therefore no longer present. Both activities are present at the same time.

### Example 2

### Preparation of Test Substances

The inventors have exemplarily prepared several peptidic substances comprising in each case specific phosphorylation sites for CDK or MAP (ERK) kinases. The test substance were as follows:

(a) CDK2 Substrates

FITC-Ahx-CMA-HHASPRK-NH₂
FITC-Ahx-CMA-HHApSPRK-NH₂
MAHHHRSPRKR-Ahx-K(FC)-NH₂
MAHHHRpSPRKR-Ahx-K(FC)-NH₂

(b) MAP Kinase (ERK) Substrates

FITC-Ahx-CMA-GGPLSPGPFK-NH₂
FITC-Ahx-CMA-GGPLpSPGPFK-NH₂
MATGPLSPGPF-Ahx-K
MATGPLpSPGPF-Ahx-K

One letter amino acid code was used; FITC=fluorescein-5-isothiocyanate, FC=fluorescin, p=phosphate, Ahx=amino hexoic acid

These test substances were phosphorylated in vitro either by cyclin A/CDK2 kinase (purchased from New England Biolabs, Beverley, Mass., USA) or by ERK kinase (Biomol, Hamburg, Germany) in kinase buffer (50 mM Hepes, pH 7.5, 10 mM MgCl₂, 1 mM EDTA, 0.01% Brij-35). The phosphorylation reaction was started by adding a solution containing ATP and magnesium (20 mM MOPS, 25 mM β-glycerole phosphate, 5 mM EDTA, 1 mM Na₃VO₄, 1 mM DTT, 75 mM MgCl₂, 0.5 mM ATP). The reaction was performed at room temperature for two hours. Subsequently, each reaction was stopped, aliquots of the reaction batch were separated on polyacrylamide gel electrophoresis (PAGE) and the test substances were visualized by UV excitation.

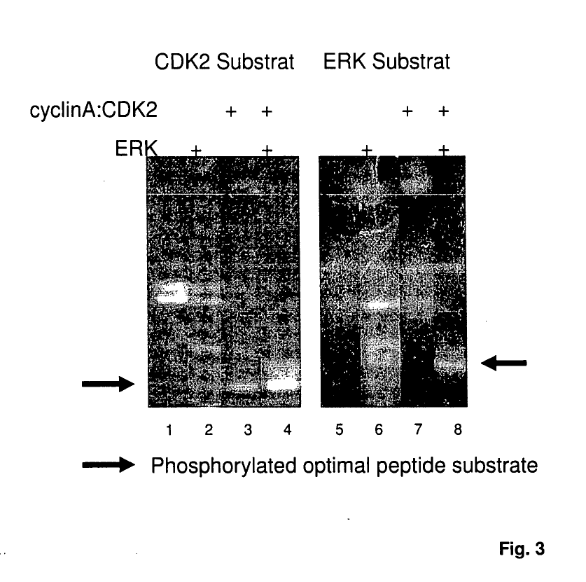

The result of one of these experiments is exemplified in FIG. 3. On the left side of FIG. 3 specific phosphorylation of the CDK2 substrate (FITC-Ahx-CMA-HHASPRK-NH₂) is demonstrated. Only in the reaction batches which contained cyclin A/CDK2 kinase the fast migrating band can be clearly observed representing phosphorylated CDK2 substrate (FIG. 3, lanes 3 and 4; arrow). The same goes for the phosphorylation of MAP kinase (ERK) substrate (FITC-Ahx-CMA-GGPLSPGPFK-NH₂). Only in the reaction batches which contained MAP kinase (ERK) the fast migrating band representing phosphorylated ERK substrate can be observed (FIG. 3, lanes 6 and 8, arrow).

The inventors have herewith provided test substances which can be specifically phosphorylated by CDK2 or ERK kinase in vitro.

### Example 3

### Preparation of the Substance According to the Invention

(a) as a Low-Molecular Weight Active Agent (“Small Molecule”)

Basically, the preparation of low-molecular weight active agents is well described in the art and ranks among the tools of a clinical chemist; cf. Böhm et al. (2002, l.c.). Especially, a large number of methods is described, by which such low-molecular active agents can be prepared, which react with signal transduction molecules such as kinase inhibitors: Buchdunger et al. (1995), “Selective Inhibition of the Platelet-Derived Growth Factor Signal Transduction Pathway by a Protein-Tyrosine Kinase Inhibitor of the 2-Phenyl-aminopyrimidine Class”, Proc. Natl. Acad. Sci. USA, Vol. 92, pages 2558 to 2562; Druker et al. (1996), “Effects of a Selective Inhibitor of the Abl Tyrosine Kinase on the Growth of Bcr-Ab1 positive Cells”, Nat. Med., Vol. 2, pages 561 to 566; Schindler et al. (2000), “Structural Mechanism for STI-571 Inhibition of Abelson Tyrosine Kinase”, Science, Vol. 289, pages 1938 to 1942. A further publication describes exemplarily for imatinib the preparation of a “small molecule”: Thomas Fischer (2002), “Der Signalhemmer Imatinib Mesilat (STI571)-Wirkprinzip und klinische Anwendung”, published by UNI-MED, Bremen, Germany. The contents of these publications are herewith incorporated into the present application by reference.

By using the methods described in before-mentioned publications the skilled person is able to prepare the substance according to the invention, without any undue burden. Starting from pre-constructed peptides as templates, small molecules can be designed by means of “molecular evolution” or “specificity evolution”, said peptides comprise segments by which a selective contacting with specific cellular kinases can occur. These segments or parts of the molecule, which derive from the peptide template, interact, for example, with the ATP binding site or the active center of the kinases. The molecule can be designed in such a way, that an activation which causes an induction of a toxicity or of a detectable signal, only occurs if an essentially simultaneous interaction with the ATP binding sites or the active centers of both kinases, i.e. the MAP kinase and the CDK2 kinase, takes place. Therefor crystal structures of the MAP kinase and the CDK2 kinase might be needed which are accessible in public databases.

(b) as a Peptide

The substance according to the invention can be prepared by means of commonly used peptide synthesis methods, resulting in the following structure: membrane permeable sequence—caspase cleavage site—linker—CDK2 substrate—linker—MAP kinase substrate—flourescein. The N terminus is situated on the left side, the C terminus is situated at the right side. A conceivable amino acid sequence reads: RRRRRRRRR-DEVD-HHASPRK-Ahx-GGPLSPGPF-Ahx-K(cf). In this representation the standardized one-letter code for amino acids is used, cf stands for carboxy-flourescein, Ahx is amino hexoic acid. This sequence can also be modified, so that the substance is activated in the case of a double-phosphorylation of both substrates, resulting in the induction of a toxicity or a detectable signal. In order to assure this result, further segments or molecules or molecule sections can be provided, which are activated by a simultaneous phosphorylation of both substrate segments of the substance.

The functioning of the substance can be verified in a mouse model. This is described in the publication of Traggiai et al. (2004), “Development of a Human Adaptive Immune System in Cord Blood Cell-transplanted Mice”, Science, Vol. 304 (5667), pages 104 to 107. By means of this model, the double-phosphorylation of the substance in transformed cells can be proved. This publication is incorporated into this application by reference.

In this model, mice with normal human immune system are generated. This model can be modified so that mice with human AML are generated, within which the double-phosphorylation of the substance according to the invention, can be shown.

Of course, other designs of the substance according to the invention, are conceivable, for example substrate segments can be designed in that way, so that a toxic activity is induced after an enzymatic conversation of the substrate segments.

The inventors have prepared several exemplary diagnostic substances according to the invention. Each of those contains two phosphorylation sites, one site was specific for CDK2, the other site was specific for the ERK. The diagnostic substances are as follows:

CDK2/MAP Kinase (ERK) Substrates

FITC-Ahx-CMA-HHASPRK-Ahx-GGPISPGPFK
FITC-Ahx-CMA-HHApSPRK-Ahx-GGPISPGPFK
FITC-Ahx-CMA-HHASPRK-Ahx-GGPIpSPGPFK
FITC-Ahx-CMA-HHApSPRK-Ahx-GGPIpSPGPFK
MAHHHRSPRKR-Ahx-TGPLSPGPF-Ahx-K(Ahx-CF)
MAHHHRpSPRKR-Ahx-TGPLSPGPF-Ahx-K(Ahx-CF)
MAHHHRSPRKR-Ahx-TGPLpSPGPF-Ahx-K(Ahx-CF)
MAHHHRpSPRKR-Ahx-TGPLpSPGPF-Ahx-K(Ahx-CF)
HHRSPRK-Ahx-GGPLSPGPF-Ahx-K(CF)
HHRpSPRK-Ahx-GGPLSPGPF-Ahx-K(CF)
HHRSPRK-Ahx-GGPLpSPGPF-Ahx-K(CF)
HHRpSPRK-Ahx-GGPLpSPGPF-Ahx-K(CF)

One letter amino acid code was used; FITC=fluorescein-5-isothiocyanate, p=phosphate, Ahx=amino hexoic acid, CF=carboxy-fluorescein. In some cases, peptides are modified by addition of the caspase cleavage site DEVD and/or nona-arginine (RRRRRRRRR).

### Example 4

### Diagnosis of a Tumor Disease by Means of the Substance According to the Invention

Blood is taken from a patient suffering from leukemia and can be, if appropriate, treated or cultivated according to methods well known in the art.

Subsequently, the blood cells are incubated with the substance obtained as described in example 3. The substance is designed in such a way, that it becomes double-phosphorylated in case of the simultaneous presence of the MAP kinase (ERK) and the CDK in the cells. In case of the presence of only one of the two kinases or of a distinct different chronological appearance of the two kinases, the substance is merely single-phosphorylated.

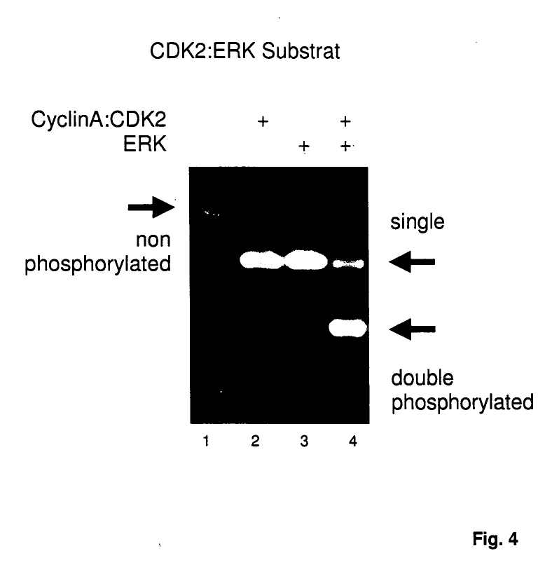

The result of one of these experiments (for CDK2/ERK substrate FITC-Ahx-CMA-HHASPRK-Ahx-GGPISPGPFK) is shown in FIG. 4. The inventors have established a tumor-cell environment by providing simultaneous activity of CDK2 and MAP kinase (ERK) kinase in vitro. In case where the diagnostic substance is in the presence of such tumor-cell environment double phosphorylation occurs (FIG. 4, lane 4, arrow, double phosphorylated), whereas in the presence of a non-tumor cell environment (solely CDK2 activity or alternatively solely MAP kinase (ERK) activity) the substance becomes merely single phosphorylated (FIG. 4, lanes 2 and 3, arrow, single). The substance remains non-phosphorylated in case where no kinase activity is present (FIG. 4, lane 1, arrow, non-phosphorylated). The different phosphorylation status of the substance are demonstrated by the migration behavior of the latter in PAGE, i.e. the double phosphorylated substance migrates faster (indicating a transformed tumor cell and allowing a positive diagnosis) than the single phosphorylated substance (indicating a non-transformed normal cell and a negative diagnosis).

Alternatively, the substance can be designed as a “biosensor” for its usage in the FRET (fluorescence resonance energy transfer) or/and quenching technology. Suitable FRET pairs, for example coumarin and fluorescein or rhodamine and fluorescein, or EDANS and Dabcyl as example for a quencher-pair, are provided, so that in case of a double-phosphorylation of the substance the conformation of the latter is changed, resulting in loss of FRET and quenching signals because of the spatial separation of the fluorescent moieties.

Another possibility is to maintain FRET signal by phosphor-dependent chymotrypsin digestion. The construction of such a substance lies within the ability of a specialist, methods suitable therefor are already commercially available in form of construction kits. An example thereof is the Z'-LYTE™ assay of the company Invitrogen (available on the website of Invitrogen). The content of the description of this assay is incorporated into the present application by reference. Here, a cleavage site for chymotrypsin is constructed N-terminal to the phosphorylation site (serine residue) of the ERK and CDK substrate. In the case where serine is phosphorylated, the site is protected and can not be cleaved by chymotrypsin. The described substance (pro-pro-drug) contains two such potential cleavage sites (the modified ERK and CDK substrates) which both can be protected by phosphorylation. Thus, no cleavage and thus sustained FRET signal can be detected exclusively in the double phosphorylated substrates. Single phosphorylated or non-phosphorylated FRET constructs are cleaved and FRET signals are lost by the spatial separation of the respective fluorescent moieties.

After the incubation the cells are lysed. The lysate is treated with protease. Afterwards, the FRET signal is read. In this connection also a usage in the FACS and a single cell profiling (cf. Irish et al. (2004), l.c.) can be carried out.

In the case of the detection of a signal or loss of the signal that indicates a double-phosphorylation, the diagnosis is positive.

The inventors have prepared several substances according to the invention which can be used in the FRET technology:

FRET CDK2/MAP Kinase (ERK) Substrates

CF-CAHHHFSPRKR-Ahx-TGPFSPGPK(amc)
CF-CAHHHFpSPRKR-Ahx-TGPFSPGPK(amc)
CF-CAHHHFSPRKR-Ahx-TGPFpSPGPK(amc)
CF-CAHHHFpSPRKR-Ahx-TGPFpSPGPK(amc)
CF-Ahx-HHFSPRK-Ahx-GGPFSPGPF-Ahx-K(amc)
CF-Ahx-HHFpSPRK-Ahx-GGPFSPGPF-Ahx-K(amc)
CF-Ahx-HHFSPRK-Ahx-GGPFpSPGPF-Ahx-K(amc)
CF-Ahx-HHFpSPRK-Ahx-GGPFpSPGPF-Ahx-K(amc)
TAMRA-Ahx-CMAHHASPRK-Ahx-GGPISPGPF-K(cf)
TAMRA-Ahx-CMAHHApSPRK-Ahx-GGPISPGPF-K(cf)
TAMRA-Ahx-CMAHHASPRK-Ahx-GGPIpSPGPF-K(cf)
TAMRA-Ahx-CMAHHApSPRK-Ahx-GGPIpSPGPF-K(cf)

One letter amino acid code was used; CF=carboxy-fluorescein, amc=Coumarin, TAMRA=tetra methyl-rhodamine, p=phosphate, Ahx=amino hexoid acid

These substances adopt a closed conformation in non-phosphorylated status. In this confirmation, the FRET pair is located in direct vicinity, resulting in the emittance of a full detectable signal [100% fluorescence]. When incubating that substance with merely one kinase, i.e. MAP kinase (ERK) or CDK2 kinase, the substance becomes single phosphorylated. This single phosphorylation results in a “half-opened” confirmation of the substance and a slow decrease of the emitted detectable signal.

In contrast, the double phosphorylated construct adopts a “full-opened” confirmation, leading to a fast decrease of the emitted detectable signal. In view of the invention as claimed, in these substances the biological or/and detectable property that is induced by the reaction with the at least two molecules which largely simultaneously appear exclusively in a transformed or/and infected biological cell corresponds to a fast decrease of the fluorescence.

Similar results can be obtained using quenching molecules. In this situation, a fluorescence signal emanates when the fluorescent dyes separate after phosphorylation.

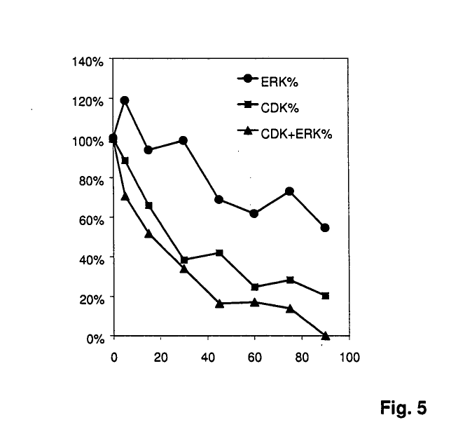

The result of such an experiment (for the construct TAMRA-Ahx-CMAHHASPRK-Ahx-GGPISPGPF-K(cf)) is depicted in FIG. 5.

At time point 0 the diagnostic substance emits 100% of the signal. 100% is defined as the quotient of emittance of rhodamine divided by the emittance of fluorescein after excitation of fluorescein. Minimal FRET signal was detected after simultaneous phosphorylation of both CDK and ERK substrates of the diagnostic substance after 90 minutes (-▴-). To the contrary, merely single phosphorylated substance still emits 20% (-▪-) or even 80% (-●-) of the initial detectable signal within that time period.

The inventors have therefore exemplarily provided several test substances that are suitable for the use as a diagnostic substance according to the invention.

Another appropriate method for preparing the diagnostic substance according to the invention, is described in Chi-Wang Lin and Alice Y. Ting (2004), “A Genetically Encoded Fluorescent Reporter of Histone Phosphorylation in Living Cells”, Angew. Chem. Int. Ed., Vol. 43, pages 2940 to 2943. The content of the publication is incorporated into the present application by reference.

1. Substance, that reacts with at least two molecules which largely simultaneously appear exclusively in a transformed and/or infected biological cell, said reaction resulting in the induction of a biological and/or detectable property.

2. Substance according to claim 1, that is a therapeutic substance.

3. Substance according to claim 1, that is a diagnostic substance.

4. Substance according to claim 1, wherein the molecules are cellular enzymes, which are involved in the regulation of the cell cycle.

5. Substance according to claim 4, wherein the cellular enzymes are kinases is a diagnostic substance.

6. Substance according to claim 1, wherein one of the two molecules is an enzyme of the ras/raf signal transduction cascade, and the other of the two molecules is an enzyme of the CDK signal transduction cascade.

7. Substance according to claim 1, that is a substrate for the at least two molecules.

8. Substance according to claim 1, wherein it comprises at least two different phosphorylation sites, one of which is the phosphorylation site for one of the two molecules, and the other is the phosphorylation site for the other of the two molecules.

9. Substance according to claim 1, that is designed in such a way, that after a reaction with at least one cellular factor the induction of the biological and/or detectable property is modified.

10. Substance according to claim 9, wherein the cellular factor is selected from the group consisting of pro-apoptotic molecules, anti-apoptotic molecules, and the telomerase.

11. Substance according to claim 1, that is designed in such a way, that an entrance or an uptake into the biological cell and/or cellular compartments is enabled.

12. Substance according to claim 1, that is a low-molecular weight active agent (“small molecule”).

13. Substance according to claim 1, that is a peptide.

14. Substance according to claim 1, wherein the design is realized by providing a segment that mediates permeability through the membrane.

15. Substance according to claim 14, wherein the design is realized by providing a sequence of arginin residues.

16. Substance according to claim 1, that is designed in such a way, that a binding to an affinity column is enabled.

17. Substance according to claim 16, wherein the design is realized by providing at least histidine residues and/or a GST tag.

18. Substance according to claim 1, whereby the biological property has a direct or indirect toxic effect on a transformed biological cell and/or an infected biological cell.

19. Substance according to claim 8, wherein the phosphorylation sites are provided within the sequence of the p53 protein or segments thereof.

20. Substance according to claim 1, that is designed in such a way, that the detectable property is detectable by means of imaging methods.

21. Expression vector, encoding a peptide according to claim 13.

22. Composition, that exclusively induces a biological and/or detectable property in a transformed and/or an infected cell.

23. Composition according to claim 22, comprising a substance that reacts with at least two molecules which largely simultaneously appear exclusively in a transformed and/or infected biological cell, said reaction resulting in the induction of a biological and/or detectable property.

24. Composition according to claim 22, comprising an expression vector encoding a peptide that reacts with at least two molecules which largely simultaneously appear exclusively in a transformed and/or infected biological cell, said reaction resulting in the induction of a biological and/or detectable property.

25. Composition according to claim 22, that is a pharmaceutical composition comprising a pharmaceutical acceptable carrier.

26. Composition according to claim 22, further comprising an activity-enhancing agent.

27. Composition according to claim 26, wherein the activity enhancing agent is selected from the group consisting of: a tumor promoter, phorbole-12-myristate-13-acetate (PMA), ionomycin, a cytostatic, an antibody, herceptin, rituximab, a growth factor, G-CSF, and FGF.

28. Method for diagnosing a tumor disease and/or an infection in a living being, comprising the following steps:
(a) providing a biological sample to be analyzed;
(b) analyzing the appearance of molecules in single cells of the biological sample, and
(c) correlating of the finding of essentially simultaneously appearing molecules in single cells of the sample, which exclusively appear in a transformed or/and infected biological cell in an essentially simultaneous manner, with a positive diagnosis.

29. Method according to claim 28, comprising performing the analysis in step (b) by means of single cell profiling or FRET technology.

30. Method according to claim 28, comprising in step (a) stimulating of the biological sample by means of a tumor promoter.

31. Method according to claim 28, wherein the tumor promoter is selected from the group consisting of phorbole-12-myristate-13-acetate (PMA), ionomycin, a cytostatic, an antibody, herceptin, rituximab, a growth factor, G-CSF, FGF.

32. Method according to claim 28, wherein step (a) includes incubating the biological sample with a substance that reacts with at least two molecules which largely simultaneously appear exclusively in a transformed and/or infected biological cell, said reaction resulting in the induction of a biological and/or detectable property, and step (b) includes detecting the detectable property.

33. Method according to claim 28, wherein the substance is a diagnostic substance that reacts with at least two molecules which largely simultaneously appear exclusively in a transformed and/or infected biological cell, said reaction resulting in the induction of a biological and/or detectable property.

34. Method for diagnosing a tumor disease and/or an infection in a human being in vitro, comprising the following steps:
(a) providing a biological sample originating from said living being, said biological sample containing single cells to be analyzed,
(b) analyzing in said single cells of said biological sample the activity of a first molecule involved in the regulation of the cell cycle and the activity of a second molecule involved in the regulation of the cell cycle, wherein said first molecule is involved in the regulation of the cell cycle at a first time in normal cells, and said second molecule is involved in the regulation of the cell cycle at a second time in a normal cell, and
(c) correlating the finding of essentially simultaneous activities of said first molecule and said second molecule in said single cells of said biological sample with a positive diagnosis.

35. Method according to claim 34, wherein:
(i) the first molecule is a retinoblastoma protein;
(ii) the second molecule is a MAP kinase, and
(iii) the biological sample is an in vitro blood sample.

36. Method for treating a tumor disease and/or an infection in a living being, comprising administering a substance according to claim 2.

37. Method for treating a tumor disease and/or an infection in a living being, comprising administering an expression vector according to claim 21.
